(19) United States
(12) Patent Application Publication
(10) Pub. No.: US 2007/0184046 A1
(43) Pub. Date: Aug. 9, 2007

(54) Use of a phospholipase A2 for the preparation of pharmaceutical and/or cosmetic compositions for the local and/or systematic treatment and/or prevention of diseases and/or processes caused by intra- and extracellular pathogens expressing membrane phospholipids

(76) Inventors: Luis A. Costa, Buenos Aires (AR); Vincente Garcia Villarrubia, Majadahonda (ES)

Correspondence Address: Leopold Presser;SCULLY, SCOTT, MURPHY & PRESSER, 400 Garden City Plaza, Garden City, NY, 11530, US

(21) Appl. No.: 11/784,110

(22) Filed: Apr. 5, 2007

Related U.S. Application Data
(62) Division of application No. 10/692,897, filed on Oct. 24, 2003, now abandoned.
Application No. 10/692,897 is a continuation of application No. PCT/ES02/00198, filed on Apr. 24, 2002.

(30) Foreign Application Priority Data
Apr. 24, 2001 (AR) P01 01 01891
Dec. 11, 2001 (AR) P01 01 05751

(51) Int. Cl. A61K 38/46

(52) U.S. Cl. 424/94.6

(57) ABSTRACT

The invention relates to the use of phospholipases, v.gr. sPLA₂, as an active agent in the production of pharmaceutical and/or cosmetic compositions for the treatment of diseases caused by micro-organisms, the envelope or membrane of which comprises phospholipids, particularly glycerophospholipids. In particular, said invention relates to the use of sPLA₂ from Crotalus durissus terrificus venom as a biocide component for the preparation of compositions for the treatment of viral infections, particularly acquired immunodeficiency states VIH-1 and VIH-2, bacterial and parasitic infections, such as infections caused by plasmodium and leishmania-type parasites.

### FIELD OF THE INVENTION

This invention relates to use of certain phospholipases, -sPLA₂, in the formulation and preparation of pharmaceutical compositions, respectively, cosmetic compositions, particularly sPLA₂-ll from the venom of Crotalus Durissus terrjficus in the formulation and preparation of compositions for the treatment of pathogenies indistinctively mediated by germs and/or animal and human modified cells, which express membrane phospholipids, such as glycophospholipids. Among such germs and cells, the following can be mainly mentioned: tumoral cells, cells transformed by intracellular pathogens and, in addition, extracellular germs, such as streptococcus and pneumococcus. Among other germs, we find viruses in general, and, particularly the human immunodeficiency virus K[V-1 and KIV-2, bacteria, such as mlcrobacterium tuberculosis and leprae and plasmodium and leishmaniasis parasites.

### BACKGROUND OF THE INVENTION

In spite of their toxicity, the venom of certain ophidian, particularly such aggressive species as Naja Nigricolis, Naja Naja atrox, Crotalus durissus terr[ficus and from toads such as Bothrops asper, etc., have been considered useful not only in the preparation of anti-ophidian serum, but also in the treatment of certain algias (1 a).

On the other hand, and as a result of searches conducted in the field of chemotherapy for treating tumors, the field of application thereof has extended as source to obtain certain fractions and drugs with unexpected specificity on cardiotherapy techniques (18), an specificity and efficacy increased by combination with other venom or fractions thereof from different sources. (2 a).

Such investigations had led to isolation and identification of factor or component involving the anti-oncogenic activity. This is a segregated 2-phospholipase, v. Gr., segregated phospholipase A₂(sPLA2)-(2), which anti-oncogenic activity has been confirmed and demonstrated by Luis A. Costa et al (3 a).—

The field of application of the sPLA₂ isolated from the venom of the above mentioned species and from native venom has been enlarged by investigations conducted by David Fenard et al (4), who have verified that certain phospholipases A₂ isolated from other vectors such as Naja Naja mossambica, Naja nigricolis, have a “potent” inhibitory activity regarding both replication and invasive capacity of HIV-1 y HIV-2 based on blocking of certain membrane receptors of human cells. It has been discovered that these lipases act on the basis of mechanisms different from anti-retroviral agents known and applied to date, and which tend to improve present treatments and to reduce the rate of resistance.

This finding and the experimental confirmation thereof offer an important innovation for the treatment and prophylaxis of viral infections mediated by HIV-1 and HIV-2. However, results thereof cannot be extrapolated, i.e. they do not authorize the conception and confirmation that all sPLA₂ have or share the inhibitory activity found by Fenard et all (4). For the case of la Naja Naja mossambica, this limitation is illustrated on page 2, lines 1 to 10 (op.cit.), a restriction repeated on page 9, lines 1 to 4 of the same publication.

Phospholipases A₂ (PLA₂) are enzymes that catalyze the hydrolysis of fatty acids from phosphoglycerides (1,2) of membranes of mammal cell and cultures (3). Two families of the -sPLA₂ (4) can be distinguished: cytosolic (cPLA2), P.M˜:85 kDa) which are associated with the metabolism of certain fatty acids: v.gr arachidonic acid (5), and segregated (-sPLA₂) (P.M.˜14-28 kDa) which are active in the extracelular medium and associated with the pathogeny of infectious/inflammatory conditions, such as: -sPLA₂-IIA y -sPLA₂-IB, which are found in sinovial and pancreatic secretions, respectively. Specifically, the -sPLA₂-II catalyze the hydrolysis of glycerophospholipids, v.gr separating the fatty acid from position 2 and the formation of the lisophospholiglycerides, which can be hydrolyzed at a next step by specific lysophospholipases and separation of the remaining fatty acid.

The scheme below illustrates the enzymatic stages that take place in biodegradation of phosphoglycerides. 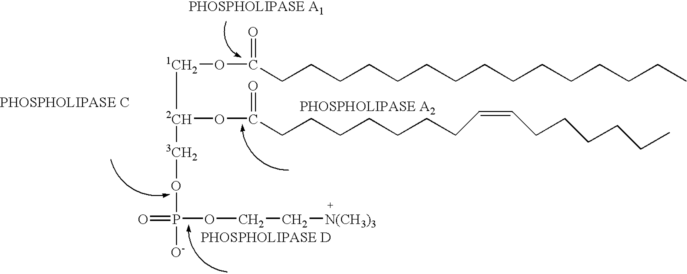
5(a)
(PHOSPHOLIPASES A₁, A₂, C, D) A₁ y A₂: fatty acids (generally A₂ is the arachidonic or oleic acid)
   R+: remainder of aminoalcohol (serine, choline, ethanolamine)
      The -sPLA₂ play an important role in several essential processes in the life of mammals, such as immunologic responses under infectious conditions, and for which reason said enzymes have been assigned a primary role in certain therapies related to the treatment of infectious pathologies. Accordingly, the sPLA₂-II would act as a bridge between natural and acquired immunity mechanisms as an activating factor for the production of IL-2-induced (mediated) IFN-γ in lymphocytes (24), and since IFN-γ induces the production of -sPLA₂ in general (25, 26), it is considered that the existence of backfeeding cycles between IFN-γ and IL-2 determines a possibly therapeutic activity for infectious conditions.

Human -sPLA₂-II are produced mainly in the liver, by a process mediated by pro-inflammatory cytokines; v.gr. Il-1 and IL-6 and the tumoral necrosis factor (TNF-α). In addition, the -sPLA₂ interact with receptors type N(3) expressed in the brain (of high affinity with certain bee venom, for example) and also receptors of the M type (expressed in the brain and lungs, respectively). The M receptors are highly homologous to the manose receptors expressed by macrophages (which cause phagocytic activation, cytokines production in macrophages), which suggests the activation of macrophages by -sPLA₂-IA (cobra venom (naja naja mossambica) and -sPLA₂-IIA). Further, said receptors constitute a physiological signal to -sPLA₂-IB and IIA.

On the other hand, there is sufficient information regarding -sPLA₂-II, which shows the importance of these enzymes in the regulation of homeostasis mechanisms in several organs in response to certain infectious, among other aggressions, for which reason -sPLA₂-II enjoy certain participation in the innate immunity (5) on the basis of the following:

1°) Large bactericidal activity of tears on account of the high -sPLA₂-II (6, 7) contents in tears (1451, 3 μg/L) (8) and in seminal fluid (15000 μg/L) (9), (values indicating antibacterial protection, possibly in combination with other bactericidal proteins, lactoferrine, etc.).

2° Presence of -sPLA₂-II produced by prostate secreting epithelial cells indicating a possibly bactericidal or viricidal function in seminal fluid.

3° Important catalytic activity of the sPLA₂ on bacterial phospholipids-non-cytotoxic-for macrophages (4), which suggests certain -sPLA₂-II incapacity , under physiological conditions, to attack “self”.

4°-sPLA₂—deficient mice are sensible to S. aureus-mediated infections, but transgenic mice for sPLA₂ have an increased resistance against the same germ (5).

Likewise, the positive role of -sPLA₂-II in certain infection-resistant mechanisms is considered possible, since patients suffering from severe infectious (peritonitis and septicemia) have significantly higher levels of -sPLA₂-II than those patients with non-infectious inflammatory events, which indicates a positive response intended to eliminate infectious agents. Further, in patients with large burns, the levels of -sPLA₂—increase only when an infection appears, which excludes the participation of factors (mediators) natural to the patient and would confirm the participation of factors inherent to the infecting agent, possibly membrane bacterial lipopolysaccharides (LPS).

This hypothetical antimicrobial role of -sPLA₂-II has been confirmed by several experiences: (i) the -sPLA₂-II of inflammatory fluids has a powerful bactericidal activity against Gram-positive bacteria (18) in response to the activity of the bacterial membrane; -sPLA₂-II demonstrated additive effects in front of beta-lactamia antibiotics used at sub-inhibitory doses (10). In addition, the following has been demonstrated: i) the in vitro and in vivo (5) bactericide power of the -sPLA₂-II against S. aureus (5); ii) the important bactericide activity of the E. coli-infected bovine serum against S. aureus, Streptococcus pyogenes and encapsulated E. coli, which can be attributed to -sPLA₂-II (11) (due to blocking by monoclonal antibodies directed against human -sPLA₂-II and restoration by addition thereof).

In brief: certain -sPLA₂-II have a natural anti-infectious activity which is not directed against all known germs: bacteria, virus, fungi and parasites; v.gr, -sPLA₂-II act directly against Gram-positive bacteria; however, the -sPLA₂-II by themselves have no germicide activity against Gram-negative bacteria, which can be attributed to their incapacity to pass through cell membranes (laminar peptideglycan web protecting membrane phospholipids) (20)); when said wall is destroyed by other host plasmatic factors present in the medium, the -sPLA₂-II are germicidally active, even at low concentrations (one of said factors being the bactericidal/permeability-increasing protein (BPD and the complex attacking the membrane belonging to the complement system.

The intervention of -sPLA₂ has been further demonstrated in cases of malaria and fingi-mediated infections, which suggests the activity of these enzymes in the pathogeny or resistance against fungicidal and parasitical infections. Since the -sPLA₂-II induce the endogenous production of the lysosomal enzyme β-glucuronidase, which is responsible for the degradation of certain intracellular parasites, the possible therapeutic activity of the -sPLA₂-II on certain infections caused thereby can be understood.

Recent in vitro experiments related to inhibitory activity of certain animal venom -sPLA₂—on HIV have shown that only 4 of 11-sPLA₂—tested seem to protect certain HIV-1(12) replication cells Said four active -sPLA₂—originated from animal venom and belonging to the group -sPLA₂-II (it is to be noted that the porcine (pancreatic IB) recombinant human ones (pancreatic, IB group and IIA group) have no antiviral effect (22). Said active -sPLA₂-II were isolated from bee and cobra venom, Naja mossambica mossambica, taipoxine y la nigexine from cobra Naja nigricollis, in addition to crotoxin from Crotalus durissus terrificus, which anti-HIV activity has been evaluated by the Applicants (13).

Based on these studies, a series of facts can be inferred which support the biological activity of the -sPLA₂:
1) The viricidal effects of the -sPLA₂—are selective. Not all -sPLA₂—have an activity against HIV, a diversity demonstrated by the following experience_The study with monoclonal antibodies directed in respect of human -sPLA₂-II has shown that there are no crossed reactions with the human pancreatic PLA₂, nor with the PLA₂ from Crotalus durissus venom, and, further the -sPLA₂-II from mammals do not interfere with the anti-HIV activity of those obtained from animals. (12).
2) Though the HIV membrane essentially comprises glycerophospholipids (main substrate from -sPLA₂), its anti-HIV activity is not caused by catalytic effects on lipids from the virus membrane, since they respond to interactions on specific receptors present in the HIV membrane.

Some phospholipases with no catalytic activity, such as the Ba IV from venom of Bothrops asper, have a weak anti-HIV effect and the addition of -sPLA₂—inhibitors have no effect on cell infection. Further the anti-HIV activity of -sPLA₂—is not caused by cytotoxic effect on the infected cells (12,13). It can be thus inferred that the -sPLA₂—having an anti-HIV activity work as specific receptors. These receptors seem to be of the N type, which are expressed in cultures and cells, including immunological ones (3). In addition, other experiences have shown that the anti-HIV effects of certain -sPLA₂—are associated with other N receptors.
3) Some tests have demonstrated that the sPLA2-II act upon biding of the virus to the cells but before release of the Reverse Transcription Complex (RTC).

It is to be noted that the -sPLA₂-II having activity against HIV do not interfere with the CD4/gp 120 binding, nor with the formation of syncytial cells, and in spite of that they strongly inhibit the entrance of the virus to cells. This latter effects, evaluated by introcytosolic detection by Gagp24, are similar to those obtained by chemiokine SDF-1 which blocks said binding. Therefore, some -sPLA₂ seem to respond to their capacity to prevent CTR dissociation from the virion on the membrane of infected cells, thus preventing this complex from reaching the cell nucleus. However, the events that take place from the moment the -sPLA₂-H bind the receptor N to prevent dissociation of the RT complex (2) are still unknown.

The Applicant has construed that the anti-HIV effects of the -sPLA₂ are related to processes fusing the virus to the host cell membrane, as it is known that the HIV-1 infection starts with interaction of the viral membrane gp120/gp41 viral complex with at least two receptors of the host cell: receptor CD4 and a member of the receptor family for chemiokines; and then, the gp120/gp41 complex in response to conformational changes leading to fusion of the viral membrane with the host cell membrane. Next, the CTR is released, which migrates to the cell nucleus carrying all the necessary information to start the pro-viral DNA synthesis, after integration into the host DNA.

The molecular bases for the specific HIV-1 tropism lie on the gp120/capacity to interact with a receptor of the chemiokine family, so that the HIV-1 strains with tropism for macrophages replicate therein and in TCDA4+ cells by binding to receptor CCR5 (they are the so called virus R5). On the contrary, the strains that show tropism for lymphocytes T replicate in lymphocytes TCD4+ through receptors of chemiokines (they are X4 virus). Generally, the R5 virus do not induce the formation of synticyal cells, while virus X4 do that.

The pathogenic and therapeutic importance of receptors for chemiokines have sufficient experimental support, since the use of chemiokines derivatives CC o CXC or of small molecules that bind different co-receptors act as HIV inhibitors.

Based on the detailed observation of the said cell fusing processes and the side effects of antiretroviral therapies, possible mechanisms can be deducted whereby some -sPLA₂—could have anti-HIV effect. The new developments on physiopathology of HIV-1 infection have evidenced that, apart from the already cited receptors, some substances, such as cholesterol, act as co-receptors for the chemiokine receptors, since destruction of the cholesterol of host cell membrane—by cyclodextrines—avoids the formation of synticyal cells and the in vitro infection by R4 virus and R5 virus (14). Further, considering affinity between -sPLA₂-II of venom from Crotalus durissus terrificus, and cholesterol (15), it can be concluded that at least part of the anti-HIV activity of said -sPLA₂-II (13) is caused by its capacity to modify-block said cholesterol co-receptors.

Another important aspect of the infection by HIV and the hypothetical role played by the -sPLA₂—is related to the function of cyclophylin A (Cyp A).This isomerase protein of the host which is present on the HIV-1 cover favors the CRT dissociation therefrom, whereby the CRT reaches the cell nucleus. Other authors have demonstrated that the CyPa favors the HIV-1 binding to host cells due to its capacity to attach to heparanes, through domains rich in basic traces for reasons similar to those binding heparin. Then the fusion of gp120/CD4 would occur. Consequently, considering the affinity of -sPLA₂—to heparanes, it is understood that these enzymes could interfere in the HIV binding to the host cell membrane. As a result, certain -sPLA₂-II from mammals or from animal venom have germicide effects on bacteria, fungi and parasites, either directly mediated by catalytic effects of the -sPLA₂-II on cell membranes, in response to their bond to membrane specific receptors. However, and in spite of the mostly possible participation of the -sPLA₂-II in the pathogeny of certain viral diseases, none of the human -sPLA₂-II studied have shown a direct activity against HrV (12). However, certain -sPLA₂-II isolated from animal venom, such as bees, Naja mossambica mossambica, Naja nigricollis, Bothrops asper (12) and Crotalus durissus terrificus (13), have a strong anti-HIV activity. Among them, and for example the venom from Crotalus durissus terrificus, positive effects are achieved against tumors from a suspected viral etiology. As already stated, the mechanisms whereby the -sPLA₂-II would act as inhibitors of HIV replication is due to their capacity to attach to cholesterol and heparanes of host cell membranes, thereby preventing the attachment of chemiokines and CypA on the virus cover. Therefore, and this being the basis of this present invention, this antimicrobial and particularly antiviral activity of certain -sPLA₂-II would be caused by already known germicidal effects (vide supra) and, in view of the extraordinary operative ubiquity and diversity of certain -sPLA₂-II, the applicants consider that some of them are closely related to the start and/or maintenance of specific immunological responses, which would make -sPLA₂-II be considered as enzymes capable of acting as bridges truly binding natural and acquired (specific) immunity mechanisms.

Regarding this hypothesis, it is evident that the -sPLA₂-II actively cooperates with the production of gamma interferon (IFN-γ) induced by interleukin 2 (IL-2) in lymphocytes (34). In turn, IFN-γ induces the production of -sPLA₂, which would be translated in the creation of a backfeeding circuit between both mediators, which lasts as far as endotoxin is present, which would explain the co-relation between the levels of both mediators during the letal sepsis caused by Gram-negative bacteria. Considering the capacity of IFN-γ to self-enlargement in sites far from origin thereof, the systemic character caused by the septic process can be understood, considering the IFN-γ in the antibacterial and antiviral resistance, and which also explains that its increase strengthens the anti-infectious resistance of the host. However, this evidences the role of IFN-γ in the launching of specific responses mediated by lymphocytes T “helper” 1 (Th1), which would clearly associate the -sPLA₂ with adapted immunity mechanisms.

Finally, there is no doubt about the important role played by the natural killer cells (NK) in host resistance mechanisms against an aggression. Since their antibacterial and antiviral activity is known, as well as its participation in antitumoral resistance mechanisms, mainly antimetastasic ones. Similarly, NK cells act as true bridges binding the innate and acquired immunity mechanisms.

The activation of NK cells is started by numerous endogenous factors, among them: the interleukin 1 (IL-1), 2(IL-2) and 12 (IL-12) and the interferon alpha (IFN-α) and gamma (IFN-γ), among other cytokines; the possible relationship between -sPLA₂—and NK cells, the fact that treatment with some -PLA₂ inductors produce an inflammatory reaction that can induce the regression of experimental glioma in rats.

In addition, the tumor regression coincides with increases in -PGE2 levels and leukotriene B4 (LTB4), as well as with the presence of NK cell infiltrates in the site of tumor regression (16), which at least suggests the capacity of -sPLA₂—or of metabolites produced thereby (PGE2 y/o LTB4) to mobilize NK cells to the tumor site. More recently, other authors have demonstrated the participation of -PLA₂ and some metabolites, mainly LTB4, in the mechanisms of cell lysis mediated by NK cells (31). In this sense, the same group describes the manner in which the occupation by receptor CD16 of NK cells causes them to activate the cPLA₂ and to secrete -sPLA₂, though both -PLA₂ do not seem to play an important role in the release of granules, which are the ones responsible for the lysis of the leptocyte (18). On the contrary, the occupation by another NK receptor (NKR-P1A) causes the same effects on the said PLA₂ (cytosilic y segregated) but with release of the granules in charge of the lytic function of NK cells (19). Finally, other authors explain this situation by showing that activation of NK cells is primarily started by the presence of lysophospholipids (lysophosphatidilcholine, arachidonic acid , or products of the lipoxygenase route) present in leptocyte membranes, which subsequently leads to activation of cPLA₂ and -sPLA₂ in NK cells with the subsequent release of granules (20). In brief, the -sPLA₂ produced by the NK cells themselves play a primary role in the mechanisms of activation thereof. However, the consulted literature contained no data showing that the exogenous addition of -sPLA₂—would be capable of activating NK cells, in this application, the -sPLA₂—from venom of Crotalus durissus durissus, among other effector lymphocytes. The data obtained from the experiences provided below sufficiently explain the said anti-infectious activity.

Based on interpretation and evaluation of experiences from the prior art, considered and evaluated in the above text and completed with the experimental work carried by the Applicants, as summarized in Example 1 and following Tables and Charts, a new application of certain phospholipases A2 is provided, specifically that of sPLA2 from venom of Crotalus durissus durissus as agent of composition for treating infectious conditions mediated by microorganisms, and which membrane comprises glycerophospholipids as structural component.

It is to be also noted that in mammals, the synthesis of phospholipids, phosphoglycerides into phospholipids, essentially occurs in the surface of the endoplasmatic reticule, and part of the synthesized phospholipids are retained on the surface of said reticular structure, but most of them migrate and the polars are inserted in the membrane structures at different proportions. The mechanism whereby synthesized phospholipids have a specific destination is not known. In addition, they are moved and oriented in vesicular form in the Golgi apparatus, a mechanism in which cytosolic proteins are also involved (Lehnning et al; op. cit).

Consequently, since both Phospholipase sPLA₂ (3a) from venom of Crotalus durissus terrificus and from the complexes formed by Crotoxin A and Crotoxin B, and if necessary in combination with the cardiotoxin of Naja naja atra have a sufficient viricidal activity to prevent contamination, v.gr, HIV-1 and HIV-2 in vitro proliferation in human cells, the applicants have considered the possibility, then experimentally justified, of using both phospholipase sPLA₂ from crotoxin and the and the from the crotoxin complex and the already mentioned combinations thereof, as anti-HIV1 and anti-HIV-2 agents.

In support of this working hypothesis, there are available data showing that the sPLA₂-II is a little high in viral infections, contrary to what happens in patients with sepsis and patients suffering from bacterial non-septic infections (6), which leads to conclude that the absence of sPLA₂ is related to certain viral escaping mechanism. Considering that the sPLA₂-II is essential for the production of interferon gamma (IFN-γ) induced by interleukin 2 (IL-2) in lymphocytes (7), and considering the role of IFN-γ in antiviral and antibacterial resistance, either directly (8) or through the induction of the so called natural killer cells (NK) (9,10), the critical function of these cells in antiviral resistance, including regarding the HIV virus, can be understood. Likewise, the IFN-γ induces the production of PLA2 (11,12), which can be translated in the creation of a back-feeding circuit, which would allow to maintain a good antiviral resistance. Further, and considering the IFN-γ capacity for self-enlargement in sites remote to its origin (13), we can infer the existence of a systemic condition of antiviral resistance. The quantitative information herein contained corresponds to the following data: 1) anti-HIV activity in vitro of the crotoxin complex and of sPLA₂ and 2) potentiating activities of the crotoxin complex and sPLA₂ on the NK cells compartment.

Abstract of the Invention

It is therefore a main object of this invention the use of a the phospholipase, particularly sPLA₂-II in the preparation of pharmaceutical and/cosmetic compositions which comprise effective amounts of said complex as active agent in combination with a biocompatible carrier, for the treatment of infectious conditions mediated by pathogenic germs that express phospholipids, v.gr, glycerophospholipids, as structural components of the membrane thereof.

It is another object of said invention the use of the crotoxin complex for the preparation of pharmaceutical and/or cosmetic compositions in effective amounts of said complex as active agent in combination with a biocompatible carrier, for the treatment of infectious conditions mediated by pathogenic germs that express phospholipids, v.gr., glycerophospholipids, as structural components of the membrane thereof.

It is a further object of this invention to use the crotoxin complex from Crotalus durissus durissus in the preparation of the said pharmaceutical and/or cosmetic compositions.

Additionally, it is another object of this invention to provide a method for the local and/or systemic treatment of infectious conditions mediated by pathogenic germs characterized by comprising treating patients suffering from an infectious conditions mediated by glycerophospholipids secreting pathogenic germs with pharmaceutical and/or cosmetic compositions comprising effective amounts of said complex.

### BRIEF DESCRIPTION OF THE DRAWINGS

FIG. 1 represents the results obtained by triple fluorescence in flow cytometry for in vitro effects of VRCTC-310-ONCO on natural human killer cells (NK) (CD3−CD56+CD4−CD8−) isolated from peripheral blood.

FIG. 2 represents the results obtained by triple fluorescence on flow cytometry of the in vitro effects of VRCTC-310-ONCO on human cytotoxic lymphocytes (LTC) (CD3+ CD56+CD4−CD8+) isolated from peripheral blood.

FIG. 3 represents the results obtained by triple fluorescence on flow cytomertry of the in vitro effects of VRCTC-310-ONCO on human lymphocytes T-helper (Th) (CD3+ CD56+CD4−CD8−) isolated from peripheral blood.—

### DETAILED DESCRIPTION OF THE INVENTION

The use of Phospholipases comprises several variations in the field of pharmacology. Among them, the -sPLA₂ from venom of Crotalus durissus durissus has already been proposed and used in the treatment of neoplasias (27,28). This present invention provides a new application of said enzymes, particularly the -sPLA₂ isolated from said venom, based on results obtained from experiments conducted by the Applicants and on the treatment of certain infections, using said enzymes as active agent.

Then the invention refers indistinctly to the use of said phospholipases -sPLA₂, for formulation and preparation of pharmaceutical and cosmetic compositions to be administered by the most suitable route and means according to the condition of patients and the characteristics of the infecting agent. Preferably, this invention refers to use of -sPLA₂ from Crotalus durissus durissus in the preparation of compositions, for example, injected even for preventive purposes. Obviously, in all cases and situations, the doses, administration schedule, etc. will depend on the condition of the patient, as well as on virulence of the pathogenic agent, seriousness of the infection, etc., which will be subject to the interpretation and decision to be made by the acting professionals. The use of the crotoxin complex and of the segregated phospholipases A₂ (-sPLA₂) which form part of said complex, according to what is herein described and to what will be hereinbelow claimed, comprises the preparation of pharmaceutical and cosmetic compositions, indistinctly, as active agent suitably carried and including excipients, diluents, etc. commonly used in the preparation of tablets, capsules, injections (intramuscular, subcutaneous, etc.), dressings, ointments, etc. In practice, the formulation and preparation of said compositions involve the use of diluents, binding or disintegrating agents, as the case may be; resorption accelerants, lubricants, etc. which are all well-known in the conventional art.

The use of liquid carriers allows to prepare formulations in the form of solution, dispersions for subcutaneous, intramuscular, pareteral administration, for example; topical compositions, including scheduled, sustained or specific organ- or tissue-directed release. In each case, the compositions , if necessary, are combined with other therapeutic agents.

The term “effective amount” as used herein refers to the doses of -sPLA₂—to be administered and capable to relieve, eliminate the symptoms of pathologic disorders associated with the permanence and/or proliferation of the pathogenic agent: HIV, respectively with the dosis of -sPLA₂—administered to reduce the pathogeny (i.e. the population of the pathogenic agent) on a scheduled basis until termination thereof.

A typical innocuous dosage, i.e. lacking any adverse side effects, is obtained by evaluating the anti-HIV activity, determining the minimal inhibitory concentration (MIC)—concentration corresponding to the inhibition point of HIV multiplication, a technique widely known by those skilled in the art.

The said evaluation can be also conducted in vivo by using a series of dosage levels by peritoneal or endovenous injections—and oral as the case may be—in mice inoculated with HIV. The activity is measured according to survival of the groups treated after death of the untreated groups.

The following examples describe experiments conducted by the applicants in order to investigate the viricidal properties of the crotoxin complex isolated from venom taken from Crotalus durissus durissus, such experiments being specifically related to the germicide activity of the crotoxin complex and sPLA2 of said complex, particular regarding microorganisms expressing membrane glycerophospholipids.

### EXAMPLE 1

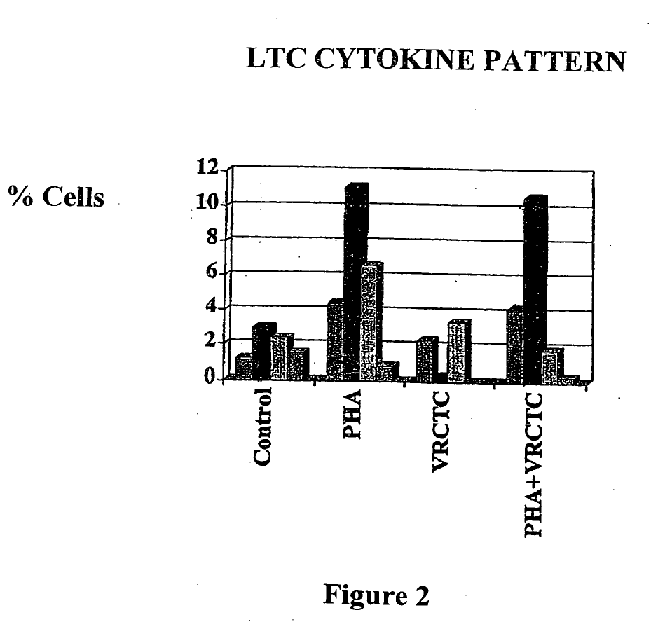

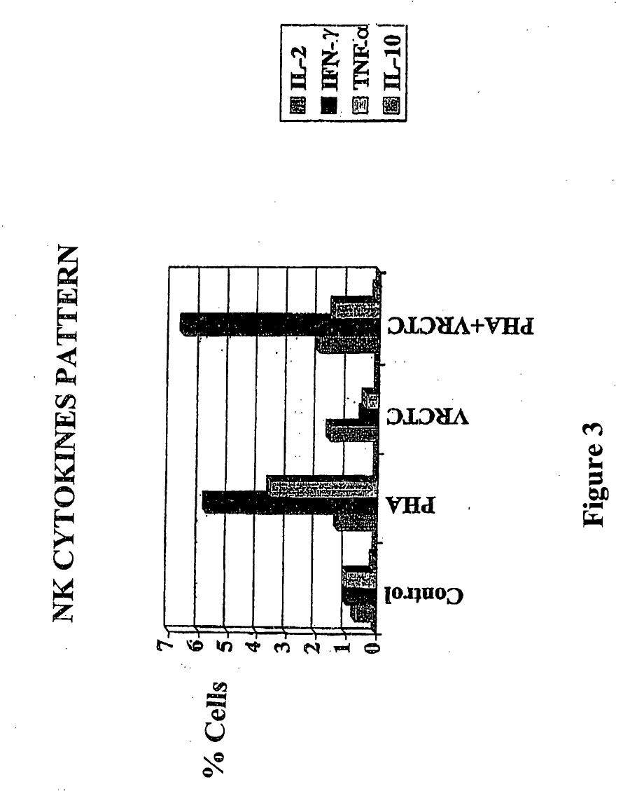

Peripheral blood lymphocytes from healthy volunteers of 18-30 years old were studied. Mononucleated cells from peripheral blood were isolated in gradient of Ficoll-Hypaque density, according to classical methods, and cultured for 48 hours in the presence of 10 μg/ml. de phytohemaglutinin (PHA), 1 μg/ml. VRCTC-310-ONCO or both. Upon activation, cells were dyed with monoclonal antibodies directed against the different markers and the expression of CD69 was determined by triple-fluorescence in flow cytometry on lymphocytes T (CD3+ CD56), cytotoxic effector cells T (CD3+ CD56+) (FIG. 3) and on natural killer cells (NK) (CD3−CD56+) (FIG. 2).

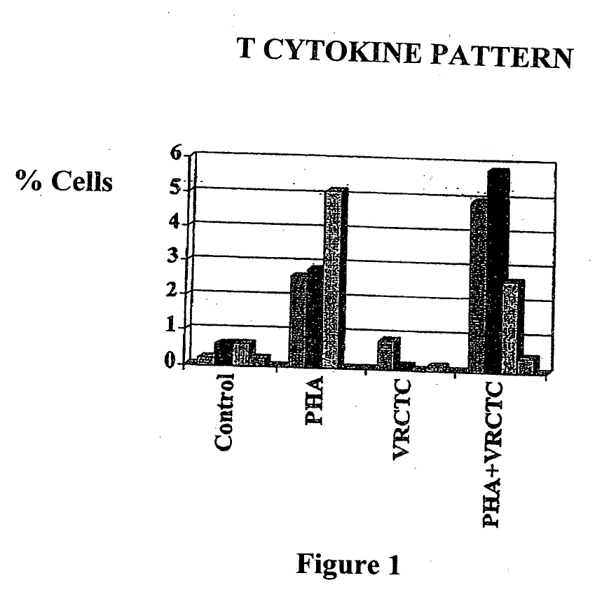

For detecting intracellular cytolines, cells were incubated with brefeldine during the last 6 culture hours and the expression of each cytokine was subsequently studied with respect to the expression of CD4 and CD8. To that effect, monoclonal antibodies were used, which were marked with FITC directed against IL-2, IFN-γ, TNF-α and IL-10, which were detected by flow cytometry (FIG. 1).

Results

Tables 1 to 3 show the results referred to expression of CD69 and the different intracellular cytokines evaluated in different lymphocyte populations under study.

TABLE 1
In vitro effects of VRCTC-310-ONCO on human Natural Killer Cells
(NK) (CD3−CD56+CD4−CD8−) isolated from peripheral blood.

| | % CELLS EXPRESSING | | | | |
|---|---|---|---|---|---|
| GROUPS | CD69 | IL-2 | IFN-γ | TNF-α | IL-10 |
| Control | 3.1 (19.6) | 0.7 | 1 | 1 | 0.1 |
| PHA | 98 (221) | 1.3 | 5.8 | 3.6 | 0 |
| VRCTC | 46.7 (61) | 1.6 | 0.5 | 0.4 | 0 |
| PHA + VRCTC | 99.4 (288) | 2 | 6.6 | 1.5 | 0.1 |

PHA: Phytohemaglutinine;
IL-2; interleukin 2;
IFN-γ; interferon gamma;
TNF-α; Tumor Necrosis Factor-alpha/cachectin;
IL-10: interleukin 10.
Data between brackets indicate MFC values

TABLE 2
In vitro effects of VRCTC-310-ONCO on human cytotoxic lymphocytes
(LTC) (CD3+CD56+CD4−CD8+) isolated from peripheral blood.

% CELLS EXPRESSING

| GROUPS | CD69 | IL-2 | IFN-γ | TNF-α | IL-10 |
|---|---|---|---|---|---|
| Control | 10.2 (76) | 1.2 | 3 | 2.4 | 1.5 |
| PHA | 95 (282) | 4.4 | 11.1 | 6.6 | 0.8 |
| VRCTC | 48.4 (196) | 2.3 | 0.3 | 3.4 | 0 |
| PHA + | 96.6 (304) | 4.2 | 10.6 | 1.8 | 0.3 |
| VRCTC | | | | | |

PHA: Phytohemaglutinin;
IL-2; interleukin 2;
IFN-γ; Interferon gamma;
TNF-α; Tumor Necrosis Factor alpha/cachectin;
IL-10: interleukin 10.
Data between brackets indicate MFC values.

TABLE 3
In vitro effects of VRCTC-310-ONCO on human lymphocytes T “helper”
(Th) (CD3+CD56−CD4+CD8−) isolated from peripheral blood.

% CELLS EXPRESSING

| GROUPS | CD69 | IL-2 | IFN-γ | TNF-α | IL-10 |
|---|---|---|---|---|---|
| Control | 0.5 (73) | 0.2 | 0.6 | 0.6 | 0.2 |
| PHA | 82 (210) | 2.6 | 2.8 | 5.1 | 0 |
| VRCTC | 6.3 (143) | 0.8 | 0.1 | 0 | 0.1 |
| PHA + | 87.6 (252) | 4.9 | 5.8 | 2.6 | 0.4 |
| VRCTC | | | | | |

PHA: Phytohemaglutinin;
IL-2; interleukin 2;
IFN-γ; interferon gamma;
TNF-α; Tumor Necrosis Factor alpha/cachectin;
IL-10: interleukin 10.
Data between brackets indicate MFC values.

Interpretation of Experimental Data

Based on experimental data contained in Tables 1 and 2 and on the comparative analysis of numerical values gathered in Table 3, it is evident that adding VRCTC-310-ONCO to the cell culture causes noticeable increases in the expression of CD69 in NK cells NK (Table 1), in cytotoxic effector T cells (Table 2) and, in general, in the whole (T) cell population (Table 3). Similarly, the addition of VRCTC slightly increases, or at least fails to interfere with, the PHA-mediated induction of CD69 (Tables 1 to 3). This is the first time the fact that a snake venom, Crotalus durissus terrificus in this case, which main active principle is a -sPLA₂, is capable of producing the in vitro expression of CD69 in human peripheral blood lymphocytes can be demonstrated.

Receptor CD69 is one of the earliest activation antigens in murine and human lymphocytes (21). The CD69 is in vitro induced on practically all cells of hematologic origin, though it is constitutively expressed in monocytes, platelets and Langerhans epidermic cells in humans. Today, it is considered that the expression of CD69 activation is a signal that predicts the lymphocyte functionality both in healthy individuals and in HIV-infected patients. In this sense, the general opinion is that the resting lymphocytes, including the T CD8+ memory, do not express or hardly express CD69. The importance of CD69 receptor in host anti-infectious resistance mechanisms has been made evident by different experimental and human models. Thus, The experimental infection by Leishmania can induce the expression of CD69 in treated patients. When that circumstance is taken together with the fact that the VRCTC acts as an inhibitor of TNF-α, intracellular expression, or when at least it does not cause the expression of this cytokine (Tables 1-3), and that this cytokine plays a disgusting role when promoting the HIV replication and NK apoptosis, the beneficial effects that could derive from a VRCTC-based treatment of AIDS and other infections involving an increased cytokine production, can be then better understood.

Finally, the behavior of cytokines as to intracellular expression under different stimuli tested, shows the same pattern in all analyzed cells (Tables and FIGS. 1-3), which evidences the VRCTC capability to activate both NK cells and T lymphocytes, particularly those having effector cytotoxic functions. Considering the fundamental role of PLA2 in the cytotoxic activity of NK cells (17-20), one should not be surprised at the effects shown by VRCTC on cytotoxic effector cells, either regarding NK or T lymphocytes.

In the light of the above detailed information, supplemented by data and conclusions derived from Example 1 and from the values summarized in the foregoing tables, it is evident that VRCTC acts in vitro as an antimicrobial agent having a double mechanism of action: (a) a direct germicide effect on the membrane of bacteria, virus and parasites; (b) an immunomodulating effect mainly based in its capacity to induce the expression of CD69 and to mitigate the production of pro-inflammatory cytokines involved in the replication of certain viruses (HIV). These consistencies and conclusions support the novelty of the object characterized in the attached claims.

1. A method for the local and/or systemic treatment of infectious conditions mediated by pathogen germs that express membranic glycerophospholipids, characterized in that it comprises treating patients showing signs of infection mediated by said pathogen germs with pharmaceutical and/or cosmetic compositions comprising effective amounts of a phospholipase A₂ segregated from a crotoxin complex obtained from the venom of Crotalus durissus terrificus.

2. The method according to claim 1, characterized in that said compositions are subcutaneous injections for anti-septic treatments.

3. The method according to claim 1, characterized in that said compositions are intramuscular injections for anti-septic treatments.

4. The method according to claim 1, characterized in that it comprises the preparation of compositions for the treatment of cutaneous infections.

5. The method according to claim 1, characterized in that said compositions are ointments, lotions and aerosols.

6. The method according to claim 1, characterized in that it comprises the preparation of ophthalmic compositions.

7. The method according to claim 6, characterized in that said ophthalmic compositions are collyria.

8. The method according to claim 1, characterized in that it comprises the preparation of compositions for the treatment of infections mediated by intracellular pathogen agents.

9. The method according to claim 8, characterized in that said infections are leishmaniasia.

10. The method according to claim 9, characterized in that the leishmaniasia comprises kala-azar, Allepo boil infantile kala-azar.

11. The method according to claim 1, characterized in that said compositions are for preventing and/or treating infections produced by HIV-1 or HIV-2.

12. The method according to claim 1, characterized in that said compositions are veterinary compositions.

13. The method according to claim 1, characterized in that said compositions comprise furthermore cardiotoxin from the venom of Naja naja atra.
